(19) United States
(12) Patent Application Publication
(10) Pub. No.: US 2018/0254097 A1
(43) Pub. Date: Sep. 6, 2018

(54) DYNAMIC MULTI-SENSORY SIMULATION SYSTEM FOR EFFECTING BEHAVIOR CHANGE

(71) Applicant: BehaVR, LLC, Elizabethtown, KY (US)

(72) Inventors: Aaron Henry Gani, Elizabethtown, KY (US); Zachary Scott Barno, Elizabethtown, KY (US); Himanshu Chaturvedi, Elizabethtown, KY (US)

(21) Appl. No.: 15/912,200

(22) Filed: Mar. 5, 2018

Related U.S. Application Data
(60) Provisional application No. 62/466,709, filed on Mar. 3, 2017.

(51) Int. Cl. G16H 20/70; G06F 3/01; G02B 27/01; G06T 19/00

(52) U.S. Cl. CPC G16H 20/70; G06F 3/015; G06T 19/003; G02B 27/017; G06F 3/013

(57) ABSTRACT

A Dynamic Multi-Sensory Simulation System for effecting behavior change includes a user interface and a sensor array for presenting information to a user and collecting biometrics from the user. The information and biometrics may be stored on a local computer or may be transmitted to and from a remote computer via a network. The remote computer may include a remote biometrics service and a dynamic experience engine. The remote biometrics service is operable to interpret biometic data signals representative of a users biological or physiological state. The dynamic experience engine is operable to present content to a user based on a user reaching a desired biological or physiological state as determined by the remote biometics services. The remote computer may also comprise a first database and a second database.

### CROSS-REFERENCES TO RELATED APPLICATIONS

This application is a Non-Provisional Application claiming priority Provisional Patent App. No. 62/466,709, filed Mar. 3, 2017, which is herein incorporated by reference in its entirety.

A portion of the disclosure of this patent document contains material that is subject to copyright protection. The copyright owner has no objection to the reproduction of the patent document or the patent disclosure, as it appears in the U.S. Patent and Trademark Office patent file or records, but otherwise reserves all copyright rights whatsoever.

### STATEMENT REGARDING FEDERALLY SPONSORED RESEARCH OR DEVELOPMENT

Not Applicable

### REFERENCE TO SEQUENCE LISTING OR COMPUTER PROGRAM LISTING APPENDIX

Not Applicable

### BACKGROUND

The present disclosure relates generally to devices, systems, and methods for influencing behavior change in humans and more particularly to devices, systems, and methods for providing multi-sensory stimuli to users in a dynamic virtual environment to influence behavior and decision-making.

It is widely known in healthcare fields that behaviors and lifestyle choices greatly impact individual health conditions. Numerous health risk behaviors such as smoking, lack of exercise, poor nutrition, tobacco use, and excessive alcohol consumption lead to higher incidences of illness and premature death. These risk behaviors also contribute greatly to obesity, type two diabetes, heart disease, stroke, cancer, and other ailments.

Although some conventional educational and therapy systems aim to inform users on behavior and lifestyle choices in an attempt to influence users and patients to make healthier decisions and daily choices, such existing systems of this nature are generally perceived by users as being overly clinical and uninteresting. This makes such systems generally ineffective at moderating and constructively influencing behavior over time.

Also, existing content platforms aiming to influence behavior and lifestyle decisions are generally not personalized to individual users, but instead include generic content distributed to various users of different backgrounds and life experiences. This “one size fits all” approach to conventional behavior change content is often ill-suited for providing effective results in patients of diverse ages and backgrounds.

Further, difficulties with financial management of physician practices is often cited as a leading obstacle to providing efficient and profitable healthcare. Much of this difficulty is related to management of chronic diseases and health problems related to lifestyle choices and risk behaviors. By better educating and influencing patients to make beneficial lifestyle choices, health outcomes will be improved and administrative and financial burdens on healthcare providers will be lessened. Healthcare providers need better platforms for assisting patients in addressing lifestyle choices and risk behaviors.

What is needed then are improvements in devices, systems, and methods for influencing behavior and lifestyle choices in users and patients.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

One aspect of the disclosure is to provide a hardware and software-based system to provide a user or patient with interactive, dynamic digital content in a simulation experience to influence behavior and lifestyle choices.

Another aspect of the disclosure is to provide a system to monitor patient feedback and/or visual activity to make dynamic content selections.

A further aspect of the disclosure is to provide a system to monitor patient biometric activity such as breathing patterns, respiration rate, muscle activity, heart rate, body temperature, heart rate variability, electrodermal activity (EDA), galvanic skin response (GSR), electroencephalogram (EEG), eye movement, and/or other physiological or psychological parameters and to make dynamic content selections and time-optimized content introduction based on the measured patient biometric activity.

Another aspect of the disclosure is to provide a system to monitor both patient feedback and patient biometric activity, and to make dynamic content selections based on the measured activity. The dynamically-selected content is provided to the user within a session via a display interface such as a computer screen, an augmented- reality headset, or a virtual-reality headset. The system further makes a determination of time-optimization to introduce the dynamically-selected content based on the patient feedback and patient biometric activity.

Yet another aspect of the disclosure is to provide a software-based dynamic content selection engine including at least one database housing numerous content packages available for dynamic selection. Over time, user data and content selection performance data is logged. The logged data is used to make future predictive enhancements to dynamic content selection.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a high level view of an exemplary embodiment of a Dynamic Multi-Sensory Simulation System.

FIG. 2 is a high level schematic view of an embodiment of a Dynamic Multi-Sensory Simulation System.

FIG. 3 is a schematic view of an embodiment of a Dynamic Multi-Sensory Simulation System.

FIG. 4 is a schematic view of an embodiment of a Dynamic Multi-Sensory Simulation System, wherein the sensor array communicates data via a network.

FIG. 5 is a schematic view of an embodiment of a Dynamic Multi-Sensory Simulation System, having a remote biometrics service and a dynamic experience engine.

FIG. 6 is a view of the various modules available in an exemplary embodiment of a Dynamic Multi-Sensory Simulation System.

FIG. 7 is an exemplary decision tree of the Dynamic Multi-Sensory Simulation System.

FIG. 8 is an exemplary display of the outside of the institute provided to a user.

FIG. 9 is an exemplary display of a welcome to the institute provided to a user.

FIG. 10 is an exemplary display of an introduction to today's module provided to a user.

FIG. 11 is an exemplary display of a motivational interview provided to a user.

FIG. 12 is an exemplary display of an avatar educational video provided to a user.

FIG. 13 is an exemplary display of a doctor educational video provided to a user.

FIG. 14 is an exemplary display of a pharmacist educational video provided to a user.

FIG. 15 is an exemplary display of a simulated fly through of a smoker's body provided to a user.

FIG. 16 is an exemplary display of a mindfulness module at the beach provided to a user.

FIG. 17 is an exemplary display of a net promoter score provided to a user.

FIG. 18 is an exemplary display of upcoming modules provided to a user.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not limit the scope of the invention. Those of ordinary skill in the art will recognize numerous equivalents to the specific apparatus and methods described herein. Such equivalents are considered to be within the scope of this invention and are covered by the claims.

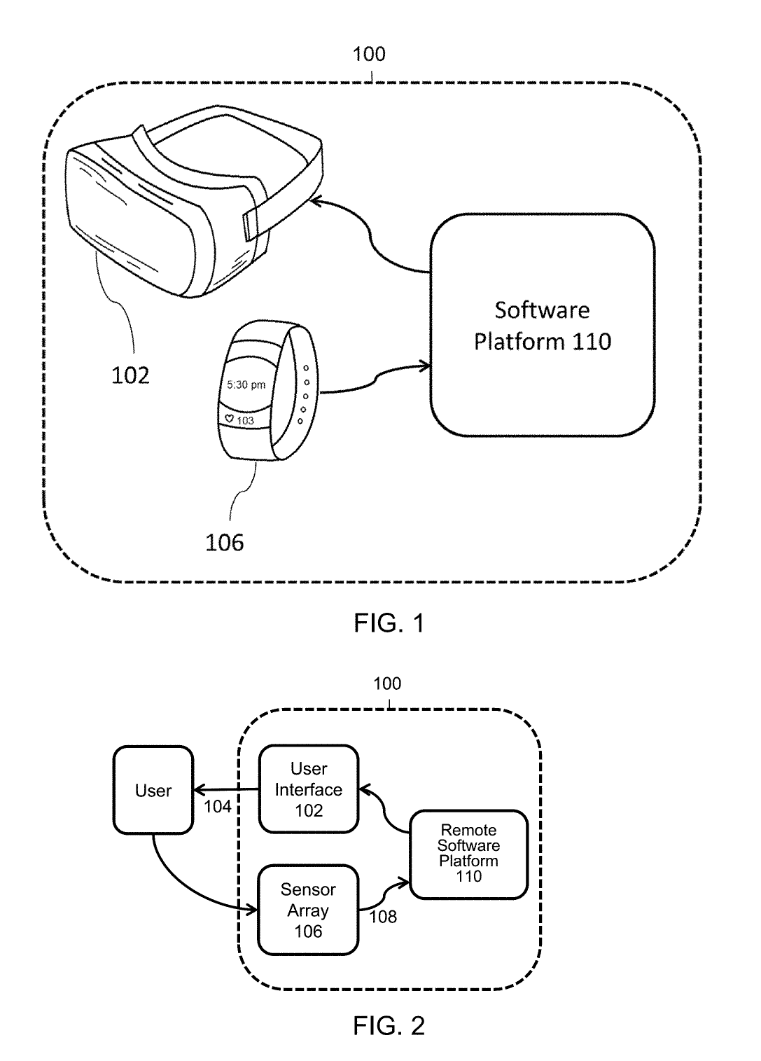

The present disclosure relates to a dynamic, multi-sensory simulation system for effecting behavior change. The system includes three main parts, an example of which is show in FIG. 1. First a user interface provides sensory simulation to a user to create a cognitive experience intended to affect the mental state of the user. Second, a sensor array provides biometric data associated with one or more physiological or mental conditions of the user. Third, a software platform receives data from sensor array and dynamically selects content to be distributed to the user via the user interface. An example is shown in FIG. 2, including a dynamic multi-sensory simulation system 100 including a user interface 102 transmitting content 104 to a user, a sensor array 106 including a data acquisition system monitoring at least one input from the user, and sending data associated with that measured input via a sensor signal 108 to a remote software platform 110 on a remote computer. The software platform 110 interprets the measured data and uses the measured data to dynamically select content and to calculate an optimized time of delivery for distribution of the selected content to the user.

User interface 102 includes any suitable display operable to provide visual or other types of content to a user. As shown in FIG. 1, an example of a dynamic multi-sensory simulation system 100 includes a user interface 102 in the form of a wearable virtual reality headset having an internal display screen positioned in a user's field of view. The user interface 102 includes an augmented reality headset or other suitable displays in some embodiments.

Sensory stimulation is provided to the user via the user interface 102. Sensory stimulation may take many forms, including visual, auditory, haptic, olfactory, gustatory, or other forms to create a cognitive experience for a user. By providing sensory stimulation, it is possible to effect the mental state of the user and to place the user into a relaxed state of mental activity such that the user may be more susceptible to selected behavior change content.

The simulations communicated to the user via the user interface 102 are generally created using devices and software to replace the normal sensory inputs the user experiences with dynamic and personalized sensory inputs that guide the user through a simulated and interactive experience. For example, a remote software platform 110 includes software configured to make dynamic selections of content for communication to the user based on various types of feedback associated with the user during a session, or obtained from prior sessions.

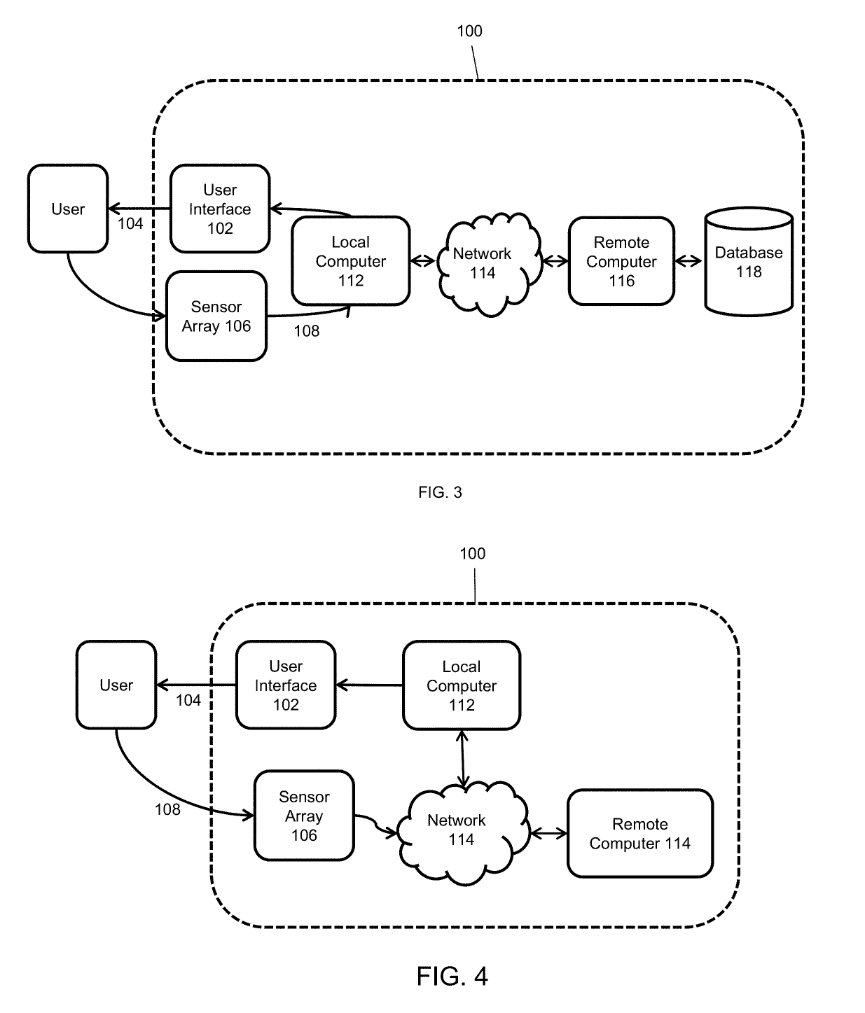

Sensor 106 may include any suitable biometric monitoring device to monitor the state of a user's body during the simulated experience. For example, sensor 106 may include biometric sensors to measure heart rate, heart rate variability, electrodermal activity (EDA), galvanic skin response (GSR), electroencephalogram (EEG), eye-tracking, body temperature, and others. As shown in an embodiment in FIG. 3, selected biometric measurements are captured via one or more sensors 106, and the associated data is either aggregated on a local computer 112 or sent over a network 114 to a remote computer. If the data is aggregated on a local computer, the data is subsequently sent over a network 114 to a remote computer 116, or server, which collects, stores and processes the measured biometric data.

Software residing on the remote computer 116 is operable to process the measured data to make a determination of what content to dynamically select from a database 118 for transmission to the user interface 102. The software residing on remote computer 116 is also operable to make a determination of when to transmit the dynamically-selected content from the database 118 to user interface 102 during a session based on the measured data. In some embodiments, the full content package including available content options to be displayed to user interface 102 is stored locally on local computer 112, and the remote computer 116 makes a determination of which selected portions of that content to send to the user interface 102. The remote computer 116 then sends an instruction of which content portions to send to the user interface 102. The remote computer 116 also sends an instruction of when to send the selected content portions based on the measured data. The measured data may also be analyzed in combination with other feedback acquired from the user, such as voice inputs or detected activity within a virtual space.

For example, during a session the sensor array 106 may detect data indicating certain content stored on database 118 should be selected and transmitted to a user to facilitate behavior change objectives. However, sensory array 106 may not yet detect an optimal physiological or mental condition for optimal effect of the content. Sensor array 106 will continue to monitor the physiological and/or mental condition of the user, and when a predetermined set of parameters is detected in the biometric data, the system will transmit the dynamically selected content via network 114 to local computer 112 and to user interface 102. Alternatively, in some embodiments, the system will send an instruction via network 114 to local computer 112 identifying a specific portion of the content stored locally on local computer 112 to send to the user interface 102. In this exemplary embodiment, the acquired biometric data may be aggregated on the local computer 112 prior to transmission to remote computer 116 as shown in FIG. 3, or data may be streamed to remote computer 116 via network 114 and subsequently aggregated and processed on remote computer 116 as shown in FIG. 4.

Referring to FIG. 3, a further embodiment provides a dynamic multi-sensory simulation system 100 for effecting behavior change. The system 100 includes a user interface 102 including a hardware display in some embodiments. A sensor array 106 includes one or more biometric sensors positioned to capture data associated with a physiological or mental condition of the user. Sensor array 106 is included in a wearable device such as a wristband, headset, vest, shirt or other suitable device in some embodiments. Additionally, in some embodiments, sensor array 106 includes an eye-tracking sensor integrated into user interface 102 such that a user may view a display and input biometric data on the same device.

User interface 102 communicates with a local computer 112 via a wired or a wireless signal path. Digital content is transmitted to user interface 102 from local computer 112 for communication to the user. Additionally, biometric data from sensor array 106 is transmitted to local computer 112. Local computer 112 communicates over a network 114 with one or more remote computers. In another embodiment, the biometric data is transmitted directly to a remote computer.

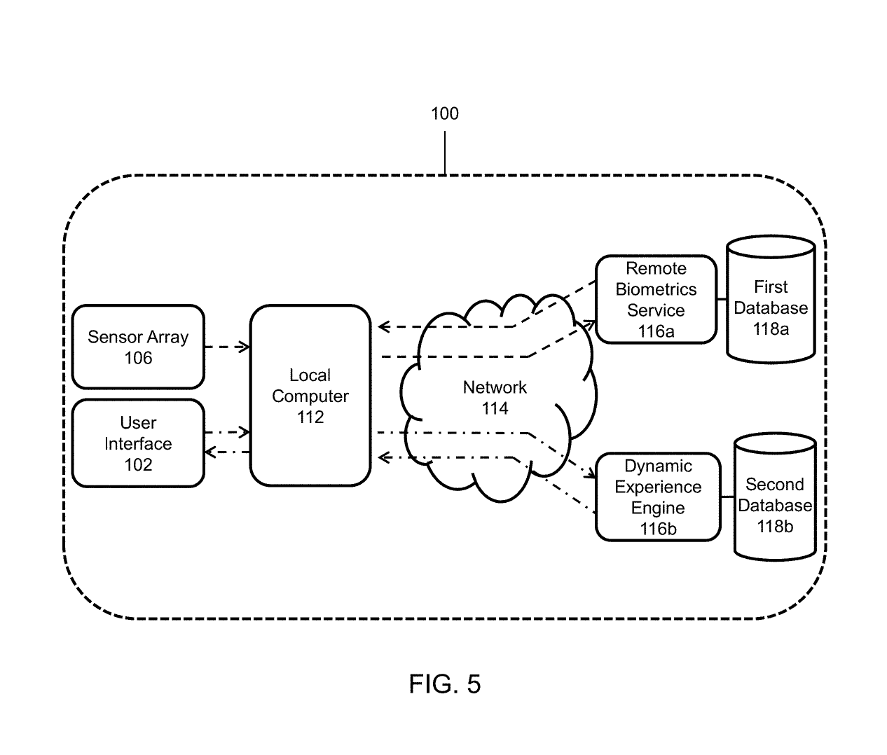

The communications signal between local computer 112 and one or more remote computers include two main components, an example of which is demonstrated in FIG. 5. First, a biometric data signal is transmitted from the local computer 112 to a remote computer having first and second programs 116a, 116b in some embodiments.

A biometrics interpretation service collects streaming or aggregated biometrics acquired from the sensor array 106 monitoring the user of the multi-sensory simulation experience. The biometric data is analyzed by a first dedicated biometrics program 116a in some embodiments, and is stored and interpreted to approximately ascertain the physiologic and/or psychologic state of the user of the multi-sensory simulation. The data may be stored in a dedicated biometrics database 118a in communication with the first dedicated biometrics program 116a. The biometrics aggregation service may summarize key biometric variables over discrete periods (for example, average heart rate for a 10 second period), and may use these raw or aggregated biometric values to compare to threshold values to determine when targeted physiologic or psychologic states may have been reached. Once the software determines a desired user state is reached, the software will instruct delivery of the dynamically-selected, personalized content to the user interface 102.

In some embodiment, the threshold values are determined in relation to data captured for each user. For example, if a user's baseline heart rate, captured at the start of the experience, starts at 80 bpm, the system determines how much the user's average heart rate declines or increases in relation to the user's baseline, by using measures of variation or change, such as standard deviation across all captured data from the user during the session. Threshold values are not limited specifically to heart rate, but any metric used to determine a user's state during a session.

In other embodiments, the threshold values are determined in relation to data captured across a population. For example, the system can either receive data associated with a population's baseline heart rate during a state of relaxation. The system determines that a user has not reached a state of relaxation based on the user's heartrate relative to the population's baseline heart rate indicative of a state of relaxation. The system may deliver content to a user once the user's heartrate has reached a threshold value based on a population's baseline heart rate during a state of relaxation. Other embodiments might include a hybrid approach, wherein the system is able to determine threshold values based on user specific values and population values.

Second, a dynamic user experience service collects log file information sent from the local computer 112 of the multi-sensory simulation machine. These log files may include one or more of: answers to questions posed to the user during the simulation, records of what virtual objects inside the simulation the user fixed their gaze on or interacted with, navigation and/or locomotion choices inside the simulation that caused the user to move around inside the simulated experience. These log files are transmitted to a second dedicated dynamic content selection program 116b, collected, stored and interpreted to ascertain elements of the user's motivation and mindset during the experience (for example, they may have answered the question of ‘why they are motivated to quit smoking’ by selecting one or more answers inside the experience). These data, combined with business rules encoded inside the dynamic user experience service and with predictive models, will be used to decide what specific content is best to deliver to the user of the multi-sensory experience at a given time. That content may then be selected from second database 118b. The dynamic user experience service may use various types of information previously collected and stored about the user and their experience, including, but not limited to: user demographic data, explicit answers to questions posed inside the experience, other physiologic or psychologic indicators which may be ascertained through passive monitoring of how they interact with the simulation.

Additionally, the simulation service computer 112 may collect various records (logs) of how the user interacts with the experience, and will store and forward this information to the dynamic user experience service 116b periodically. The dynamic user experience service 116b will send messages to the simulation service computer 112 instructing it on what content to deliver when to the user. Such content includes explicit descriptions of computer generated stimuli, which may include computer graphic simulations of people, places or things, video recordings of the real world, audio content (music, voice, sounds), or other simulations of the real world.

In many embodiments, a user may interact with a front-end software application, or Physician Control Panel or Administrative Control Panel. The front-end application or remote biometrics services 116a record biometric data captured from sensor array 106, including one or more devices connected to or worn by the patient. The biometric data is captured in data packets and streamed via network 114 in some embodiments. In some embodiments, the sensor array 106 and front-end software application, including associated data acquisition hardware, may be programmed to different data acquisition sampling rate. In some embodiments, the sensor array 106 is configured for a data acquisition sampling rate of once every sixteen seconds. In other embodiments, the sensor array 106 is configured for a data acquisition sampling rate of once every 160 milliseconds. The sampling rate is adjustable. The front-end application collects the data in a local database on local computer 112. In other embodiments the sensor array 106 directly transmits the biometric data to the remote service 116a over the network 114. The collected biometric data may be transmitted via network 114 at a programmable transmission frequency. In some embodiments, the data is transmitted at 1 Hz, or once per second. The data is transmitted via network 114 to a remote server 116 on which first and second programs 116a, 116b are stored. In alternative embodiments, the data is transmitted to more than one remote server. For example, in some embodiments a first remote server houses first program 116a and accesses first database 118a, and a second remote server houses second program 116b and accesses second database 118b.

The front-end software application on local computer 112 or the sensor array 106 may perform analysis of the acquired biometric data prior to transmission over network 114. For example, in some applications, the software is programmed for the front-end software application to calculate the mean of the biometric data every ten seconds for the prior ten second interval. The calculated data is sent via network 114 to the remote computer 116. The back end server 116 then calculates a moving average of the mean and standard deviation of a predetermined number of previous “n” iterations of the biometric summaries. In some embodiments, the back end server 116 calculates a moving average of the mean and standard deviation of the previous five transmitted biometric summaries.

When a user begins a simulation session that is dynamically-driven by the acquired biometric data, the remote computer 116 sets baseline values of the average and standard deviation of the “n” most recent biometric summaries. As the simulation experience continues, the back end server calculates a moving average of the “n” most recent summaries, and compares the moving average examples to the baseline values. When a target differential is met (for example: Moving Average Heart Rate<[Baseline Heart Rate−[0.5*Baseline Standard Deviation]]) the back end server sends a signal via application programming interface (API) to the simulation experience computer 112 that the patient has achieved the targeted biometric state, and is ready for the delivery of behavior-influencing content. This type of example calculation may be used to determine when to send the dynamically selected content to a user based on the acquired biometric data.

All the time intervals such as frequency of collecting, storing, and sending biometrics data to the back end server 116, are configurable on the back end server 116 in some embodiments. Also the number of data points that will be aggregated to evaluate the above condition is configurable. The mathematical condition used above is a preliminary hypothesis, subject to change based on the results gathered over time.

At the start of a patient session at interface 102, an operator collects information in one of two methods, or both. Either a) the operator asks the patient questions, and enters the information manually into the Physician Control Panel or Administrative Control Panel application on the local computer 112 or remote computer 116; or b) the front-end application or remote computer 116 retrieves information electronically via an API connection to the office practice management system or electronic medical records database; or c) a combination of both methods is used. The information captured is demographic information such as name, age, gender, ethnicity, etc. or condition related information such as disease state, success/failure of prior attempts at behavior change, etc., or both. This demographic and condition related information is sent to the back end server 116 where it is continually stored.

As a simulation experience commences, and during the simulation experience, data is collected in several ways. Log files are collected on the local computer 112, which record patient actions inside the simulation experience, such as navigational choices, what tagged virtual objects were examined (i.e. looked at) or interacted with by the user, and these log files are sent to the back end server 116 for storage.

The patient is also asked questions while inside the simulation experience, and responses to these questions (which may be captured by way of digital interfaces inside simulation enabling answers to be chosen (i.e. multiple choice)), or by way of voice recording from a microphone that is part of the VR head mounted display or worn on the person of the patient) are recorded.

Biometric values are captured via one or more sensors on sensor array 106, which are used as indicators of physiological or psychological arousal or relaxation, for example, during the experience.

All of these three types of data are captured and stored continually. Patient success at achieving desired behavior changes are evaluated by asking patients about their success and readiness to change inside the simulation experience, and also by follow-up outside of the simulation experience. All data collected about patient success is recorded in the same persistent data store as the other patient data.

The system then utilizes a variety of statistical learning & analytical techniques to evaluate which simulation experiences for which types of patients (types being indicated through analysis of demographic data) have the best outcomes in terms of desired behavior changes. The techniques utilized include but are not limited to: logistic regression, linear regression, linear discriminant analysis, K-Nearest Neighbors classification, Decision Trees, Bagging, Random Forests, Boosting, and Support Vector Machines.

Referring further to FIG. 5, in one embodiment, the entire sequencing of the elements experienced inside the simulation experience is driven by a workflow in the back-end server (the Dynamic Experience Engine or ‘DXE’) 116. The front end Virtual Reality Experience (the ‘VRX’) on the user interface 102 and local computer 112 is a thin client which does not store or decide on any particular sequence of actions to be taken. Instead, local computer 112 interprets the commands sent to it from the DXE software on remote computer 116 and takes appropriate action. The workflow definitions consists of states, content, transitions, and conditional logic. States define what action is supposed to be taken at a particular moment in the VRX at the local computer 112. Each state can be associated with some content (i.e., images, videos, audio tracks, animations, etc.) that are to be presented to the user. Transitions define the sequence of states from the beginning to the end of the VRX. At a particular point in the workflow a state could have options to transition to one of multiple states. The decision as to which state will follow next is made using pre-defined conditional logic.

An example of this conditional logic may look like (but is not limited to):
IF condition A is true: State 1 should be followed by State 2.
OTHERWISE: State 1 should be followed by State 3.

The conditional logic could be dependent on multiple factors such as the actions the user has taken in the current VRX session or in any previous VRX sessions, demographics data about the user or predictive models using biometrics, demographics and user interaction data. Thus, the system has the capability to provide personalized content to different users based on complex analysis.

After processing the actions of each state, the VRX makes a request via API to the DXE software 116b on remote computer or server 116 to get the next state it should transition to and the content it should present. This continues until the VRX is instructed by the DXE software 116b that the last state has been reached and to exit the program.

The workflow is defined for all possible instructions that are available at any time during any session. An instruction describes what should happen during the session, including, but not limited to displaying content. In one embodiment, the front- end application (VRX) makes a request to the DXE 116b for instructions that the VRX needs to process. The VRX repeatedly makes requests to the DXE 116b for new instructions as the VRX finishes processing the instructions already delivered from the DXE 116b. The instructions are conditional and are evaluated by an in-house rules engine which is part of the DXE 116b. The rules engine is defined using various technologies, including, but not limited to SQL statements, stored procedures, functions and web service methods. The conditions can be evaluated on any data in the system (biometrics, user input, demographic information, etc.).

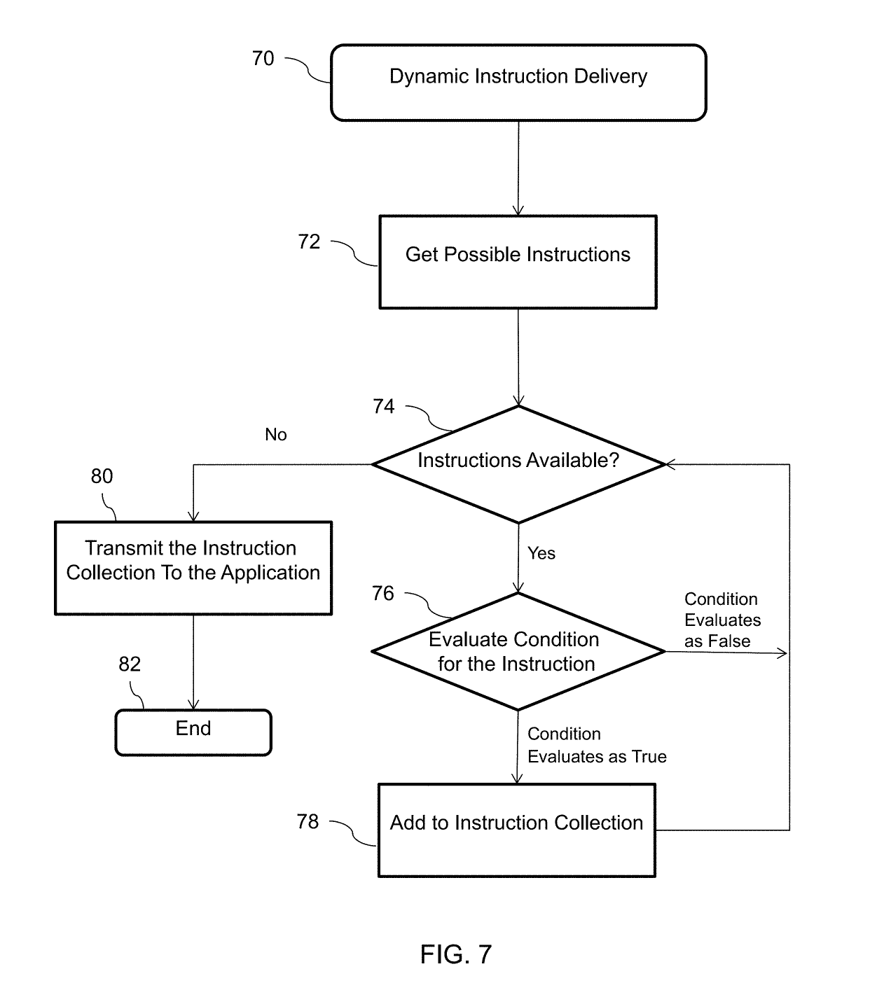

FIG. 7 demonstrates an exemplary decision tree of the system 100 when requesting instructions from the DXE 116b. The VRX makes a request for dynamic instruction delivery 70 to receive possible instructions 72. The system 100 then determines if instructions are available 74. If instructions are available, the system 100 evaluates condition for the instruction 76. If the condition is evaluated as true, the system 100 is operable to add to instruction collection 78. The system 100 is them operable to transmit the instruction collection to the application 80. The progression ends 82 after the instruction collection is transmitted to the application. If no instructions are available 74, the system 100 will end the progression of instruction delivery. If the evaluation of the condition for the instruction is evaluated as false, the system 100 will inquire again to see if an instruction is available. The system will repeat until there is no instruction available. Once the system 100 has determined that the condition for the instruction is present and the instruction is added to the instruction collection, the system 100 will loop to determine if any instructions are available. Thus, the system 100 continuously sends inquiries for instructions, wherein the instructions are only delivered when a condition for the instruction is verified. In some embodiments, when evaluating for a condition, the system will evaluate a missing condition as always being true. For example, in the case of an instruction with no rules associated with the instruction, the instruction will always be delivered.

An exemplary embodiment of the Dynamic Multi-Sensory Simulation System includes a user interface 102, a sensor array 106, a software platform 110. Information is presented to the user via the user interface 102, the user's reaction to the information is recorded by the sensor array 106, and the software platform determines subsequent information to present to the user based on the user's reaction. The system 100 is operable to present a therapy session to the user based on inputs recorded from the user. A therapy session may consist of modules.

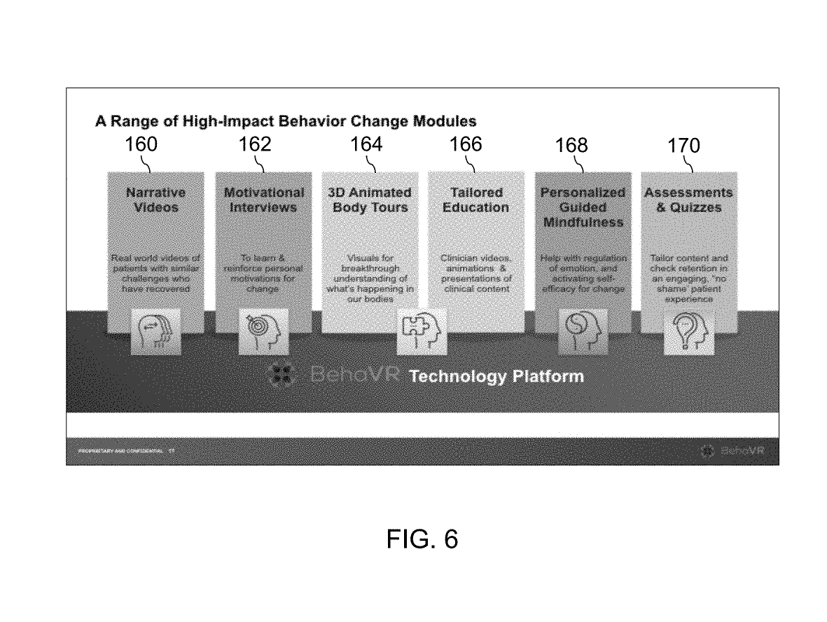

As seen in FIG. 6, the modules include narrative video module 160, motivational interview module 162, 3D animated body tour module 164, tailored education module 166, personalized guided mindfulness module 168, and assessment module 170. The narrative video module 160 includes real world videos of patients with similar challenges who have recovered. Motivation interview module 162 includes content for educating the user and for reinforcing personal motivations for change. The 3D animated body tour module 164 includes content for visualization for understanding what is happening inside of a body as a result of the undesired behavior. The tailored education 166 module includes content presented by clinicians, animations, and other various forms for presenting clinical information and content relating to the undesired behavior. The personalized guided mindfulness module 168 includes content for assisting, encouraging, and fostering regulation of emotion and activation of self-efficacy for change. The assessment module 170 includes content for verification of knowledge retention.

The various modules include content of the types shown in FIG. 6. The system presents different content (animations, films, visuals, etc.) to the user, and may capture and store different information from the user consistent with the type of content being presented. In an exemplary embodiment, in the assessment module 170, the user's answers are captured, stored, and interpreted. In another exemplary embodiment, in the personalized mindfulness module 168, the user's biometrics are captured, and interpreted. Each of these captured data are then further used for personalization or, in the case of biometrics, assessing the patient's state of relaxation and optimizing the timing of presenting certain mindfulness content.

In one exemplary embodiment, a session for smoking cessation is provided. The session begins with an Avatar welcoming the user and continues with walking the user through numerous pieces of content as well as gathering data. Potentially, a session could be any combination of educational videos, audio tracks, animations, or mindfulness exercises. In this exemplary embodiment of smoking cessation, the program includes ten modules which are structured as five knowledge modules and five mindfulness modules which are delivered alternately. A knowledge module typically consists of one or more of the following sections: (1) Motivational interviewing (e.g., Why does the user smoke, why does the user want to quit smoking, etc.), (2) Educational videos (e.g., harmful chemicals in cigarette smoke, effect of smoking on different parts of the body, etc.), and (3) Animations (e.g., short animated story about how quitting smoking can impact their lives). A mindfulness module typically consists of a user selecting the virtual location (e.g., a beach in Maldives and open green fields in Germany) and their guide (e.g., a male or female guide) for mindfulness followed by guided audio tracks. A module typically ends by describing what the users can expect in the upcoming modules as well as gathering user experience data like Net Promoter Score.

An exemplary embodiment of a module in which a physiological state triggers specific content delivery is provided. The mindfulness module in the session begins with trying to make the user calm and comfortable by lowering the user's heart-rate. The lowering of the user's heart-rate may be achieved by using a specific set of audio scripts. As long as the desired heart rate drop is not achieved, audio scripts from this set are repeatedly delivered to the user.

An exemplary embodiment of a module in which a user interactions with the system trigger specific content delivery is provided. Prior to launching the mindfulness module, a user is asked to choose the virtual location where they would like to practice mindfulness. Based on this choice, the appropriate 360 video or a 3D environment is delivered to the user.

In other embodiments, the system may further provide for various programs including content tailored for effecting specific behavioral changes. The system can be used for treatment of any suitable undesirable behavior or condition. The system may implement the following programs for: smoking, obesity, diabetes, pain management, lower-back pain recovery, pain neuroscience education, medication adherence, surgical peri-operative program, addiction recovery, COPD management, hypertension management, and cognitive behavioral therapy-based interventions for anxiety, obsessive compulsive disorder, post-traumatic stress disorder, and phobias.

Numerous other configurations for executing the disclosed system and method may be achieved, and the illustrations and description provided herein provide an exemplary embodiment. The overall system is operable to utilize biometric data in combination with user feedback during a real-time simulation session to dynamically select behavior-change content optimized for the user, and the system further assesses the biometric data in combination with the user feedback during a real-time simulation session to optimize the optimal time to present the dynamically-selected content to the user to have the greatest effect. The dynamically-selected content will vary from user-to-user, and by utilizing a virtual-reality or augmented-reality interactive user interface, it is possible to present the dynamically-selected content at an optimal time within a session in a profound and engaging way to better influence behavior and lifestyle decisions in users.

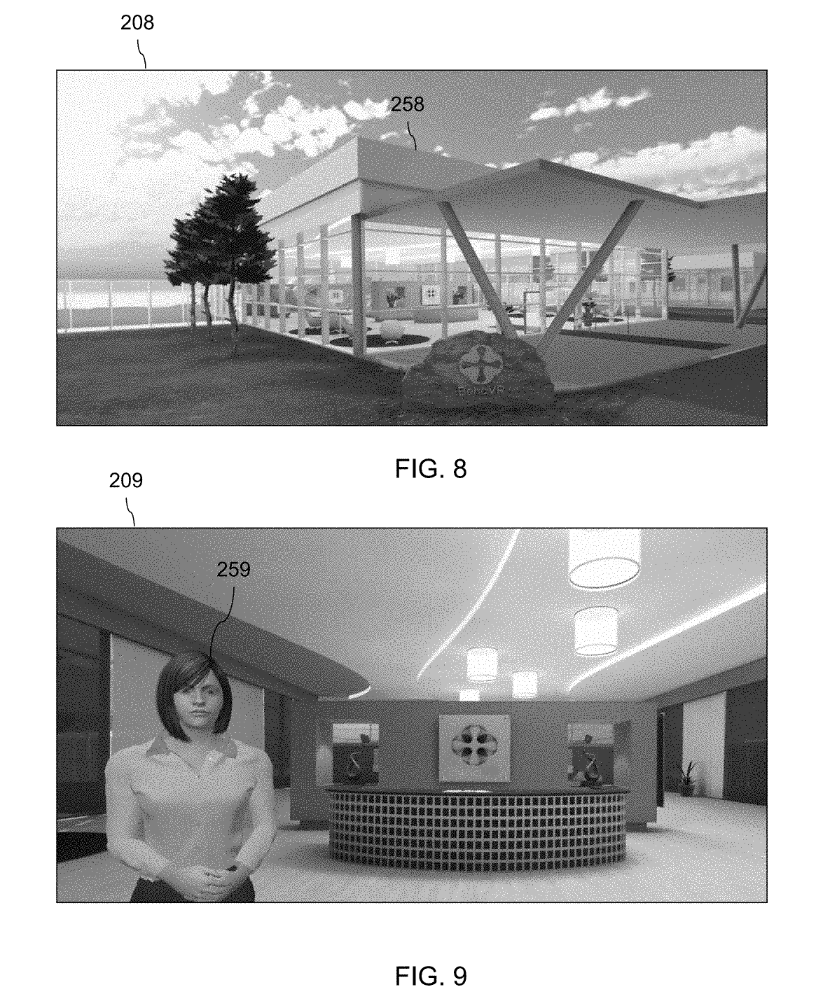

Included in FIG. 8-FIG. 18 are exemplary interfaces or screen shots of content presented to a user via the user interface 102. FIG. 8 is an exemplary display provided to a user of the outside of the institute 208. The system 100 is operable to display a virtual institute 258 is which a user enters and is able to progress through the virtual experience.

FIG. 9 is an exemplary display provided to a user of a welcome to the institute 209. The interior of the virtual institute 258 is shown in this exemplary embodiment. The interior of the virtual institute may in some exemplary embodiments display to a user an avatar 259 which guides the user through the virtual experience.

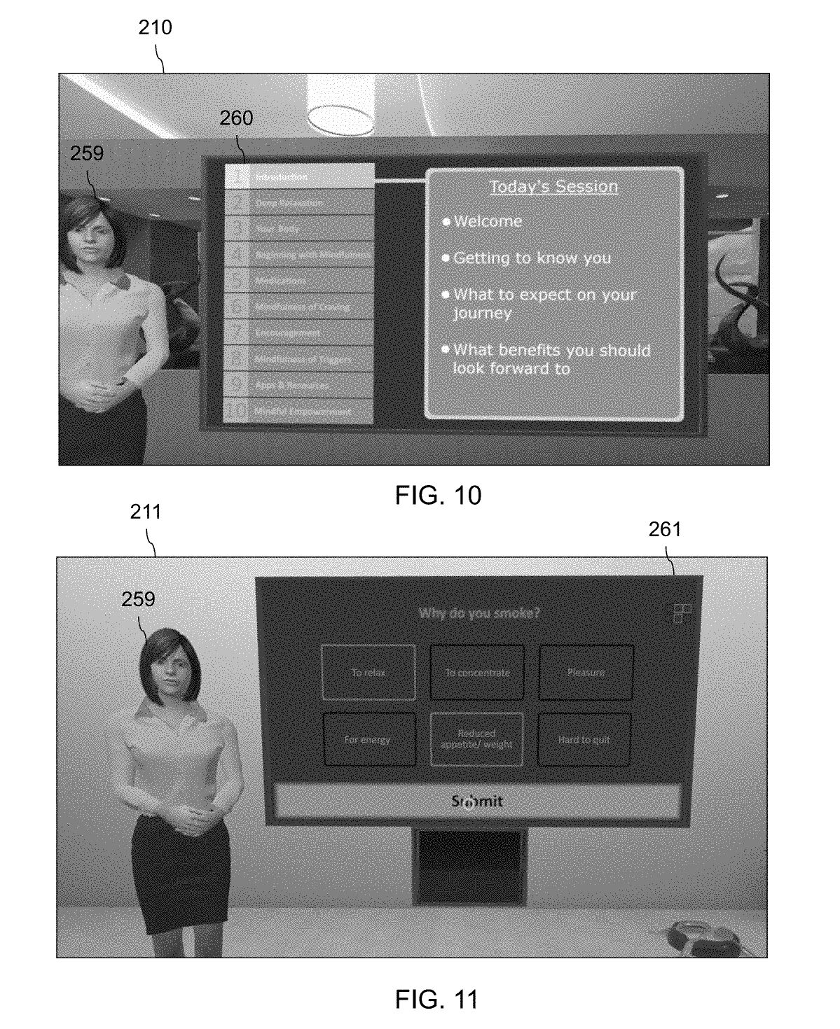

FIG. 10 is an exemplary display provided to a user of an introduction to today's module 210. In this exemplary embodiment, an avatar 259 takes the user through an introduction of the modules through which the user will progress during a virtual experience. Part of the introduction may include an introduction menu 260 displaying all of the various modules.

FIG. 11 is an exemplary display provided to a user of a motivational interview 211. This exemplary display is a representation of an avatar 259 presenting questions to a user to help the user understand why the user exhibits certain behaviors. The exemplary display may include a question and answer menu 261 which presents to the user with various selections which the user chooses in response to a posed question or scenario.

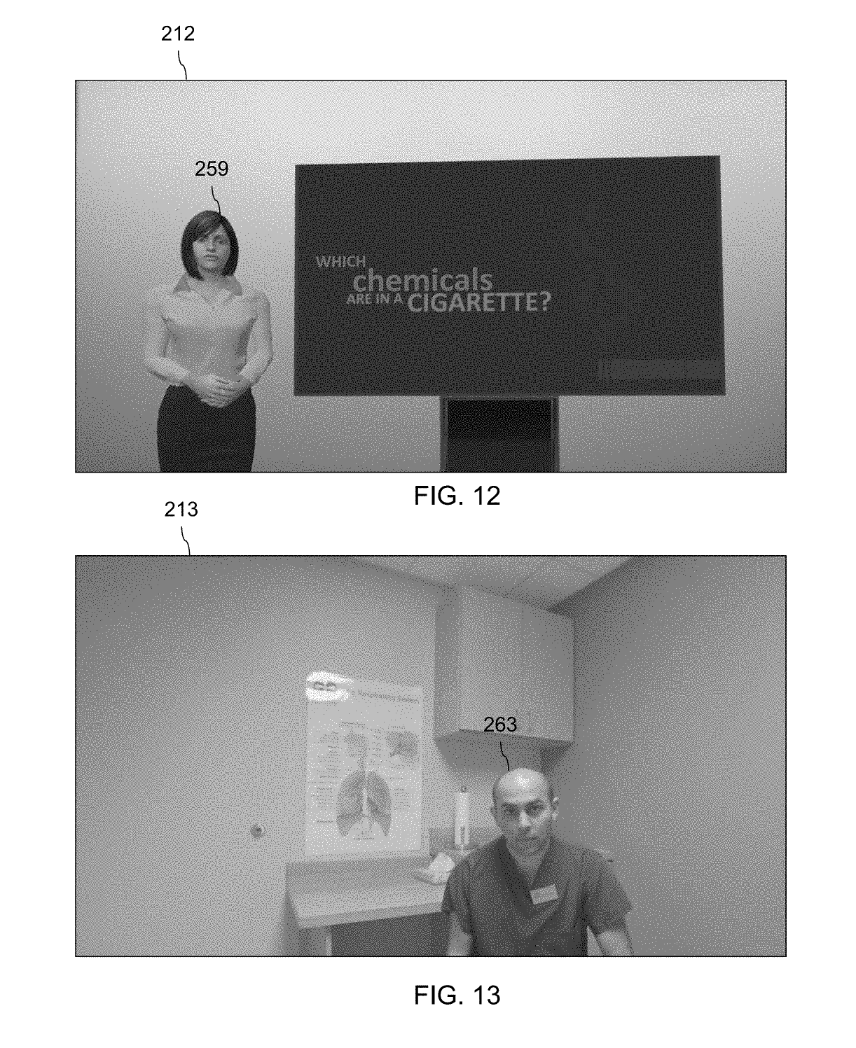

FIG. 12 is an exemplary display provided to a user of an avatar educational video 212. In this exemplary display an avatar 259 presents various educational videos and content to the user.

FIG. 13 is an exemplary display provided to a user of a doctor educational video 213. In this exemplary display, a video is presented to the user in which a doctor 263 is educating the user on information relating to the behavior which the user is attempting to change.

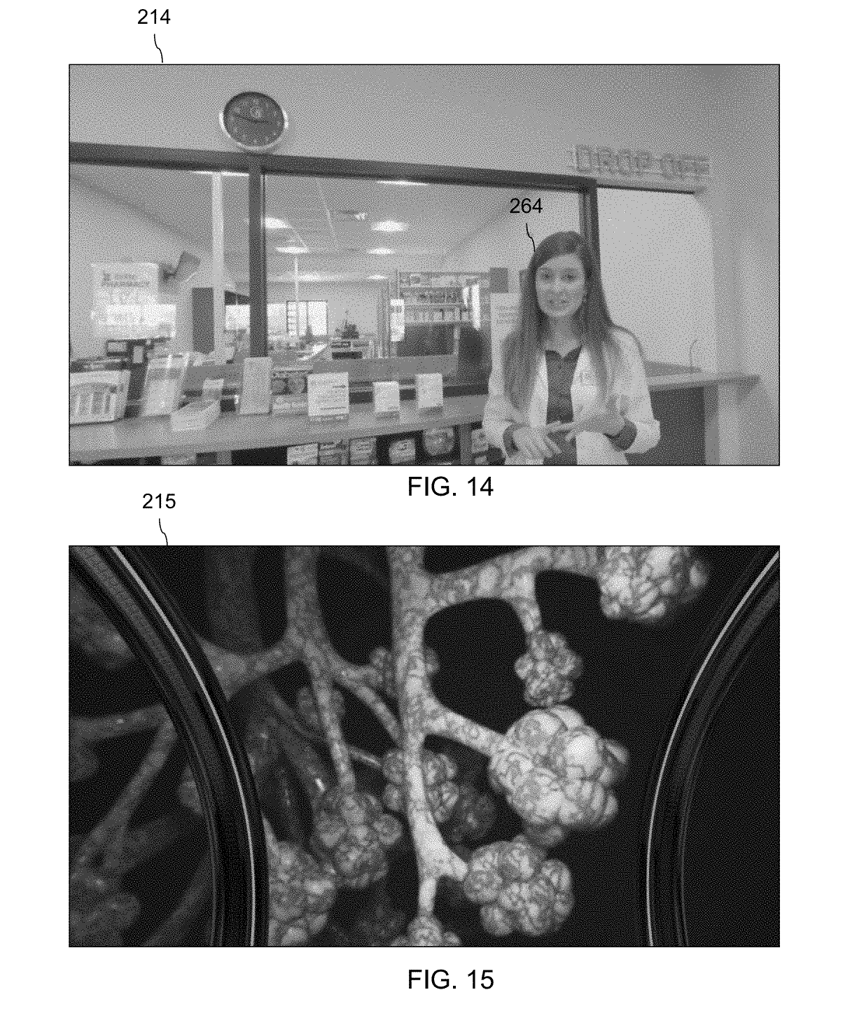

FIG. 14 is an exemplary display provided to a user of a pharmacist educational video 214. In this exemplary display, a video is presented to the user in which a pharmacist 264 is educating the user on information relating to the behavior which the user is attempting to change.

FIG. 15 is an exemplary display provided to a user of a simulated fly through of a smoker's body 215. In this exemplary display, the system 100 take the user on a virtual or simulated tour of the user's body and specifically displays to the user the effects the behavior is having on the user's body. In this exemplary display, the user is shown the effects of smoking on the respiratory system and the bronchioles.

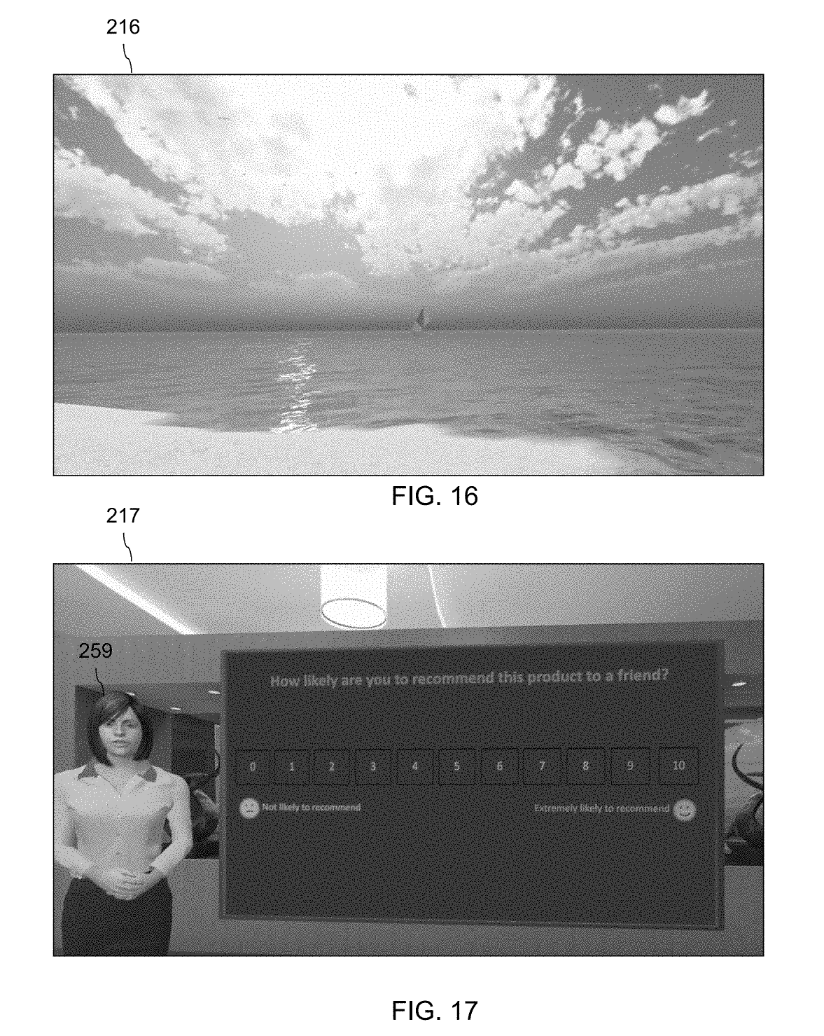

FIG. 16 is an exemplary display provided to a user of a mindfulness module at the beach 216. In this exemplary display, a user is able to meditate at a selected location, as a portion of the mindfulness module. The system 100 displays to the user the virtual location.

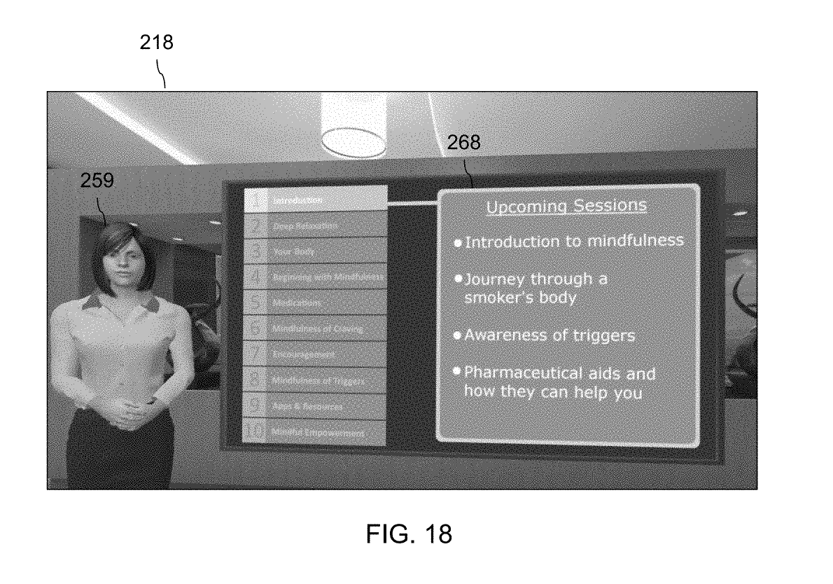

FIG. 17 is an exemplary display provided to a user of a net promoter score 217. In this exemplary display, an avatar 259 takes a user through a questionnaire relating to the virtual experience.

FIG. 18 is an exemplary display provided to a user of upcoming modules 218. In this exemplary display, an avatar 259 displays an upcoming modules menu 268 to the user for the user to understand what future session or virtual experiences will include.

Thus, although there have been described particular embodiments of the present invention of a new and useful DYNAMIC MULTI-SENSORY SIMULATION SYSTEM FOR EFFECTING BEHAVIOR CHANGE, it is not intended that such references be construed as limitations upon the scope of this invention.

What is claimed is:

1. A dynamic multi-sensory simulation system for effecting behavior change, comprising:
a user interface configured to display content to a user during a simulation session;
a biometric sensor array adapted for interfacing with the user to acquire one or more biometric data signals associated with a physiological or psychological state of the user;
a local computer configured to distribute the content to the user interface and to acquire the biometric data from the biometric sensor array; and
a remote computer connected to the local computer via a network, the remote computer including a program operable to receive and analyze the biometric data,
wherein the remote computer is configured to dynamically make a selection of content from a database during the session based on the received and analyzed biometric data, and
wherein the remote computer is configured to dynamically calculate an optimal time during a session to introduce the selected content to the user interface via the network.

2. The dynamic multi-sensory simulation system for effecting behavior change, of claim 1, further comprising a dynamic experience engine present on the remote computer.

3. The dynamic multi-sensory simulation system for effecting behavior change, of claim 2, wherein the dynamic experience engine provides workflow for a simulation session.

4. The dynamic multi-sensory simulation system for effecting behavior change, of claim 3, wherein the workflow is selected from the group consisting of states, content, transitions and conditional logic.

5. The dynamic multi-sensory simulation system for effecting behavior change, of claim 1, further comprising a remote biometrics service present on the remote computer.

6. The dynamic multi-sensory simulation system for effecting behavior change, of claim 5, wherein the remote biometrics service is capable of analyzing the biometric data signal and establishing a baseline value biometric data signal.

7. The dynamic multi-sensory simulation system for effecting behavior change, of claim 6, wherein the remote biometrics service is capable of detecting certain biological and physical states based on an updated biometric data signal relative to the baseline value biometric data signal.

8. The dynamic multi-sensory simulation system for effecting behavior change, of claim 1, wherein the biometric sensor array is selected from the group consisting of a heart rate sensor, a heart rate variability sensor, a electrodermal activity sensor, a galvanic skin response sensor, a electroencephalogram sensor, an eye-tracking sensor, microphone, and a thermometer.

9. The dynamic multi-sensory simulation system for effecting behavior change, of claim 1, wherein the user interface includes a virtual reality headset.

10. A method of effecting behavior change by providing a dynamic multi-sensory simulation system, comprising:
displaying, by a user interface, content to a user during a simulation session;
collecting, by a biometric sensor array adapted for interfacing with the user, biometric data associated with a physiological or psychological state of the user;
establishing, by a processor, a baseline biometric value of the biometric data over a period of time;
determining, by the processor, when a user reaches a targeted user state based on the biometric data relative to the baseline biometric value; and
delivering to the user interface dynamically-selected content based on the desired user state.

11. The method of effecting behavior change by providing a dynamic multi-sensory simulation system, of claim 10, wherein the dynamically-selected content is selected from the group consisting of states, content, transitions and conditional logic.

12. The method of effecting behavior change by providing a dynamic multi-sensory simulation system, of claim 10, wherein the biometric sensor array is selected from the group consisting of a heart rate sensor, a heart rate variability sensor, a electrodermal activity sensor, a galvanic skin response sensor, a electroencephalogram sensor, an eye-tracking sensor, microphone, and a thermometer.

13. The method of effecting behavior change by providing a dynamic multi-sensory simulation system, of claim 10, wherein the user interface includes a virtual reality headset.

14. A kit apparatus for a simulation experience for effecting behavior change, comprising:
a user interface configured to display content to a user during the simulation experience;
a biometric sensor array adapted for interfacing with the user to record biometric data;
a second sensor adapted to record user action data based on user interactions with the content inside the simulation experience;
a local computer configured to distribute the content to the user interface and to acquire the biometric data from the biometric sensor array and user action data from the second sensor;
a biometrics interpretation service connected to the local computer, the biometrics interpretation service operable to receive and analyze the biometric data; and
a dynamic content service connected to the local computer, the dynamic content service operable to receive and analyze the user action data,
wherein the local computer is configured to dynamically make a selection of content from a database during the session based on the received and analyzed biometric data and the received and analyzed user action data, and
wherein the local computer is configured to dynamically calculate an optimal time during a session to introduce the selected content to the user interface via the network.

15. The kit apparatus for a simulation experience for effecting behavior change, of claim 14, wherein the dynamic content service provides workflow for a simulation session.

16. The kit apparatus for a simulation experience for effecting behavior change, of claim 15, wherein the workflow is selected from the group consisting of states, content, transitions and conditional logic.

17. The kit apparatus for a simulation experience for effecting behavior change, of claim 14, wherein the biometrics interpretation service is capable establishing a baseline value biometric data and a baseline value user action data.

18. The kit apparatus for a simulation experience for effecting behavior change, of claim 17, wherein the biometrics interpretation service is capable of detecting certain biological and physical states based on the received and analyzed biometric data relative to the baseline value biometric data and the received and analyzed user action data relative to the baseline value user action data.

19. The kit apparatus for a simulation experience for effecting behavior change, of claim 14, wherein the biometric sensor array is selected from the group consisting of a heart rate sensor, a heart rate variability sensor, a electrodermal activity sensor, a galvanic skin response sensor, a electroencephalogram sensor, an eye-tracking sensor, microphone, and a thermometer.

20. The kit apparatus for a simulation experience for effecting behavior change, of claim 14, wherein the user interface includes a virtual reality headset.

21. A dynamic multi-sensory simulation system for effecting behavior change, comprising:
a user interface configured to display content to a user during a simulation session;
a biometric sensor array adapted for interfacing with the user to acquire one or more biometric data signals associated with a physiological or psychological state of the user;
a local computer configured to distribute the content to the user interface; and
a remote computer connected to the local computer and the biometric sensor array via a network, the remote computer including a program operable to receive the biometric data signals from the biometric sensor array and analyze the biometric data signals,
wherein the remote computer is configured to dynamically make a selection of content from a database during the session based on the received and analyzed biometric data signals, and
wherein the remote computer is configured to dynamically calculate an optimal time during a session to introduce the selected content to the user interface via the network.

22. The dynamic multi-sensory simulation system for effecting behavior change, of claim 21, further comprising a dynamic experience engine present on the remote computer.

23. The dynamic multi-sensory simulation system for effecting behavior change, of claim 22, wherein the dynamic experience engine provides workflow for a simulation session.

24. The dynamic multi-sensory simulation system for effecting behavior change, of claim 23, wherein the workflow is selected from the group consisting of states, content, transitions and conditional logic.

25. The dynamic multi-sensory simulation system for effecting behavior change, of claim 21, further comprising a remote biometrics service present on the remote computer.

26. The dynamic multi-sensory simulation system for effecting behavior change, of claim 25, wherein the remote biometrics service is capable of analyzing the biometric data signal and establishing a baseline value biometric data signal.

27. The dynamic multi-sensory simulation system for effecting behavior change, of claim 26, wherein the remote biometrics service is capable of detecting certain biological and physical states based on an updated biometric data signal relative to the baseline value biometric data signal.

28. The dynamic multi-sensory simulation system for effecting behavior change, of claim 21, wherein the biometric sensor array is selected from the group consisting of a heart rate sensor, a heart rate variability sensor, a electrodermal activity sensor, a galvanic skin response sensor, a electroencephalogram sensor, an eye-tracking sensor, microphone, and a thermometer.

29. The dynamic multi-sensory simulation system for effecting behavior change, of claim 21, wherein the user interface includes a virtual reality headset.
